(19) United States
(12) Patent Application Publication
(10) Pub. No.: US 2023/0335290 A1
(43) Pub. Date: Oct. 19, 2023

(54) SYSTEM AND METHODS FOR CONTINUOUSLY ASSESSING PERFORMANCE OF PREDICTIVE ANALYTICS IN A CLINICAL DECISION SUPPORT SYSTEM

(71) Applicant: Etiometry Inc., Boston, MA (US)

(72) Inventors: Michael F. McManus, Pembroke, MA (US); Dimitar V. Baronov, Weston, MA (US); Robert Hammond-Oakley, Somerville, MA (US)

(21) Appl. No.: 18/134,189

(22) Filed: Apr. 13, 2023

Related U.S. Application Data
(60) Provisional application No. 63/330,554, filed on Apr. 13, 2022.

(51) Int. Cl. G16H 50/70; G16H 50/30

(52) U.S. Cl. CPC G16H 50/30; G16H 50/70

(57) ABSTRACT

A method determines an internal state variable. The method receives patient data and a model of an internal state variable. The internal state variable is calculated using the patient data and the model of the internal state variable. Gold standard data corresponding to the internal state variable is received. A statistical performance assessment of the model of the internal state variable is performed. The method determines whether a performance of the model of the internal state variable is above a prescribed threshold. A source of inconsistent data negatively impacting the performance of the model of the internal state variable is determined.

### PRIORITY

This patent application claims priority from provisional U.S. Pat. Application No. 63/330,554, filed Apr. 13, 2022, entitled, “System and Methods for Continuously Assessing Performance of Predictive Analytics in a Clinical Decision Support System,” the disclosure of which is incorporated herein, in its entirety, by reference.

### GOVERNMENT SUPPORT

This invention was made with government support under R44HL117340 awarded by the National Heart, Lung, And Blood Institute of the National Institutes of Health. The government has certain rights in the invention.

### FIELD OF THE INVENTION

Illustrative embodiments of the invention generally relate to systems and methods for patient monitoring and, more particularly, illustrative embodiments relate to continuous assessment of patient monitoring.

### BACKGROUND OF THE INVENTION

Practicing medicine is becoming increasingly more complicated due to the introduction of new sensors and treatments. As a result, clinicians are confronted with an avalanche of patient data, which needs to be evaluated and well understood in order to prescribe the optimal treatment from the multitude of available options, while reducing patient risks. One environment where this avalanche of information has become increasingly problematic is the Intensive Care Unit (ICU). There, the experience of the attending physician and the physician’s ability to assimilate the available physiologic information have a significant impact on the clinical outcome. Hospitals that do not maintain trained intensivists around the clock experience a 14.4% mortality rate as opposed to a 6.0% rate for fully staffed centers. It is estimated that raising the level of care to that of average trained physicians across all ICUs can save 160,000 lives and $4.3 Bn annually. As of 2012, there is a shortage of intensivists, and projections estimate the shortage will only worsen, reaching a level of 35% by 2020.

The value of experience in critical care can be explained by the fact that clinical data in the ICU is delivered at a rate far greater than even the most talented physician can absorb, and studies have shown that errors are six times more likely under conditions of information overload and eleven time more likely with an acute time shortage. Moreover, treatment decisions in the ICU heavily rely on clinical signs that are not directly measurable, but are inferred from other physiologic information. Thus, clinician expertise and background play a more significant role in the minute to minute decision making process.

### SUMMARY OF VARIOUS EMBODIMENTS

In accordance with an embodiment of the invention, a method determines an internal state variable. The method receives patient data and a model of an internal state variable. The internal state variable is calculated using the patient data and the model of the internal state variable. Gold standard data corresponding to the internal state variable is received. A statistical performance assessment of the model of the internal state variable is performed. The method determines whether a performance of the model of the internal state variable is above a prescribed threshold. A source of inconsistent data negatively impacting the performance of the model of the internal state variable is determined.

The method may also generate a list of potential associated error conditions causing the inconsistent data from the source. In some embodiments, the method takes corrective action to reduce an error condition causing the inconsistent data from the source. In various embodiments, a statistical performance assessment of the model of the internal state variable is performed after corrective action is taken. The method may repeat the steps of performing the statistical performance assessment, determining whether performance is above the prescribed threshold, and determining the source of inconsistent data.

Among other things, the model of the internal state variable is based on retrospective data. As described herein, the internal state variable may be a particular health event. Additionally, or alternatively, the internal state variable may be a particular patient variable, such as a patient biomarker. In some embodiments, the internal state variable may be a hidden internal state variable.

The method may determine that the source of error is a patient characteristic, and/or a patient characteristic when used with a particular sensor.

In accordance with another embodiment, a system determines an internal state variable. The system includes a risk based patient monitoring engine configured to calculate an internal state variable using patient data and a model of the internal state variable for a patient. A retrospective database has gold standard data corresponding to the internal state variable. The system also has a performance assessor configured to perform a statistical performance assessment of the model of the internal state variable. The system may also be configured to determine whether a performance of the model of the internal state variable is above a prescribed threshold. Additionally, the system may be configured to determine a source of inconsistent data negatively impacting the performance of the model of the internal state variable.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a clinical patient environment in accordance with illustrative embodiments of the invention.

FIG. 2A schematically shows details of a system in accordance with illustrative embodiments of the invention.

FIGS. 2B-2C show screenshots of a notification generated by the system in accordance with illustrative embodiments of the invention.

FIG. 3 shows a process of determining the performance of a predictive analytics system and correcting causes of error for the predictive analytics system in accordance with illustrative embodiments of the invention.

FIG. 4A schematically shows a ROC curve that may be used as a comparative performance assessment threshold in accordance with illustrative embodiments.

FIG. 4B schematically shows a ROC curve generated by the performance assessor using data from the patient database 105.

FIG. 4C schematically shows an example of a histogram of mis-labeled blood gas data.

FIG. 4D schematically shows an updated ROC curve 400D after correcting causes of error in accordance with illustrative embodiments.

### DESCRIPTION OF ILLUSTRATIVE EMBODIMENTS

In illustrative embodiments, a system provides risk-based patient monitoring of individual patients to clinical personnel. The system streams data from a plurality of medical devices that are attached/coupled with a patient. Such data streams may include, for example, pulse oximetry data from a pulse oximeter. The system also has access to data from other medical devices, electronic health records, blood labs and/or other labs, bedside monitors and hospital information systems. The various sources of data are continuously processed to calculate a risk index that predicts a score indicating the likelihood of a particular event (e.g., a soon to happen heart attack) or other patient variable/biomarker (e.g., may predict patient data that otherwise requires an invasive test using non-invasive data measurements), generally referred to as patient internal state variables.

Illustrative embodiments may be embodied as a decision support system that prompts the user with specific actions according to a standardized medical plan, when patient-specific risks pass a predefined threshold (e.g., the determination of the patient-specific risk may be determined using the model of the internal state variable). Various embodiments provide a performance assessment of the predictive indexes (e.g., for the internal state variable). Furthermore, when the performance is low, illustrative embodiments determine the cause of the error, and may take corrective action.

Additionally, some embodiments may collect “gold standard” data, or another referenced accurate value, as well as streamed medical device/sensor data. The gold standard data collected from a large patient population may be used to determine errors for a particular sensor given specific variable conditions. For example, it may be that a specific type of patient (e.g., race, age, weight, diagnosis, procedures) under particular conditions (e.g., above a given heart rate) causes error in the measurements of a given sensor 102. Additionally, or alternatively, it may be the case that the type of hospital, and/or geographic location of the medical care facility are correlated with a particular error in a sensor. The system then correlates, over time, the “gold standard” data, data produced by the device being tested, and the other information listed above to determine the cause of the error condition.

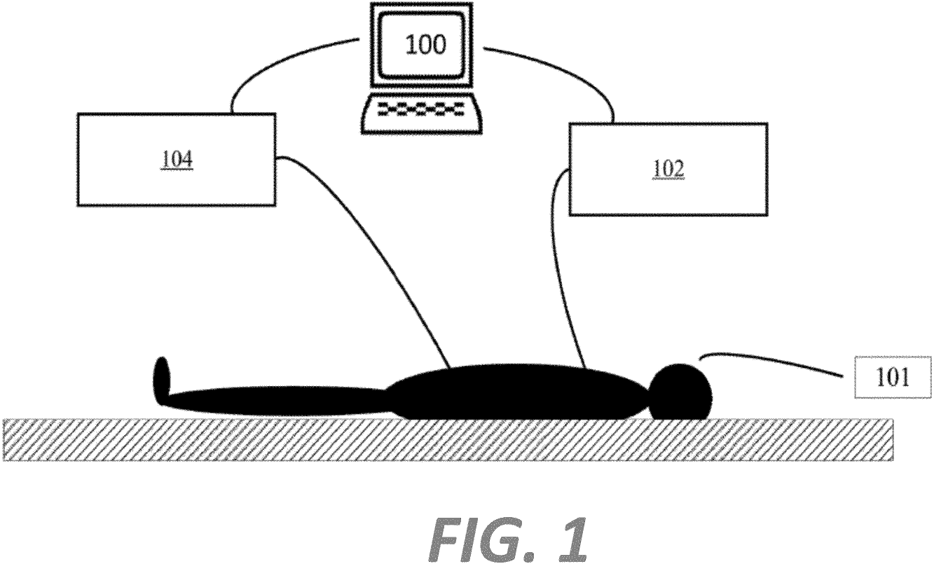

FIG. 1 schematically shows a clinical patient environment in accordance with illustrative embodiments of the invention. The environment includes medical devices having sensors 102 (including bedside monitors 102) for providing patient data to health providers, such as physicians, nurses, or other medical care providers. To that end, a patient 101 may be coupled to one or more physiological sensors 102 or bedside monitors 102 that may monitor various physiological parameters of the patient. It should be noted that a patient 101 may be a human, or not human (i.e., a non-human, e.g., a dog).

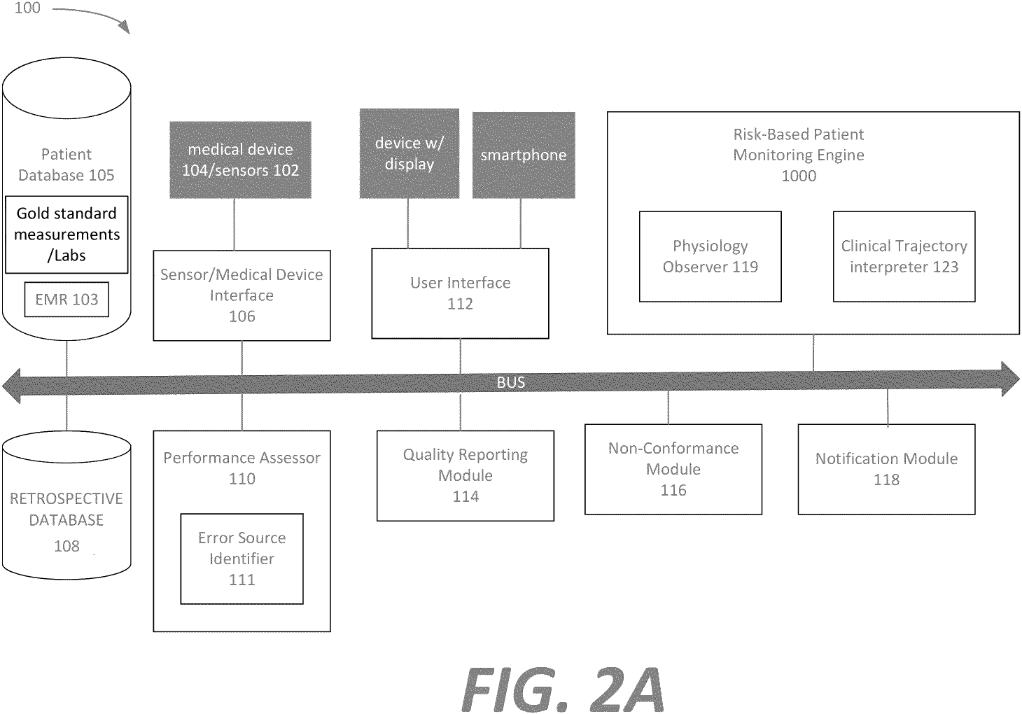

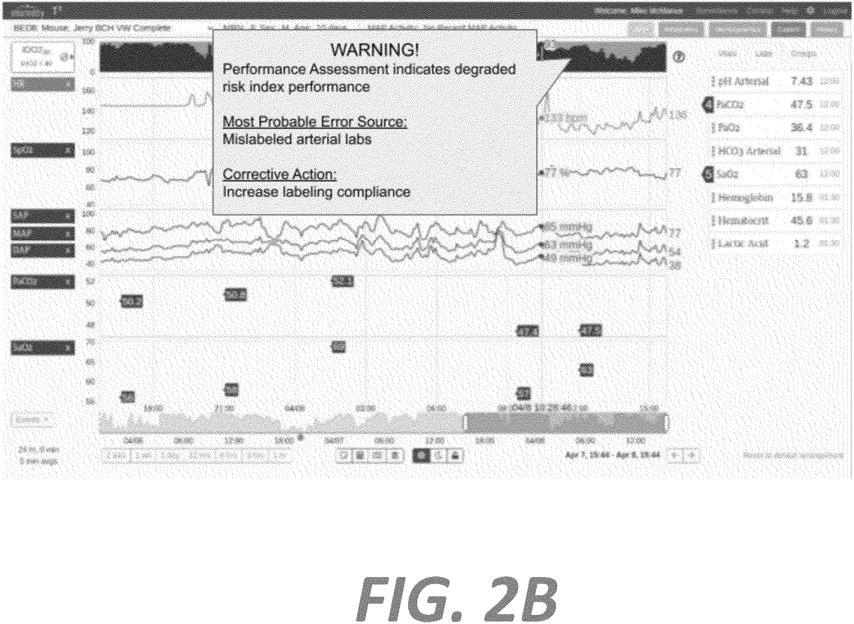

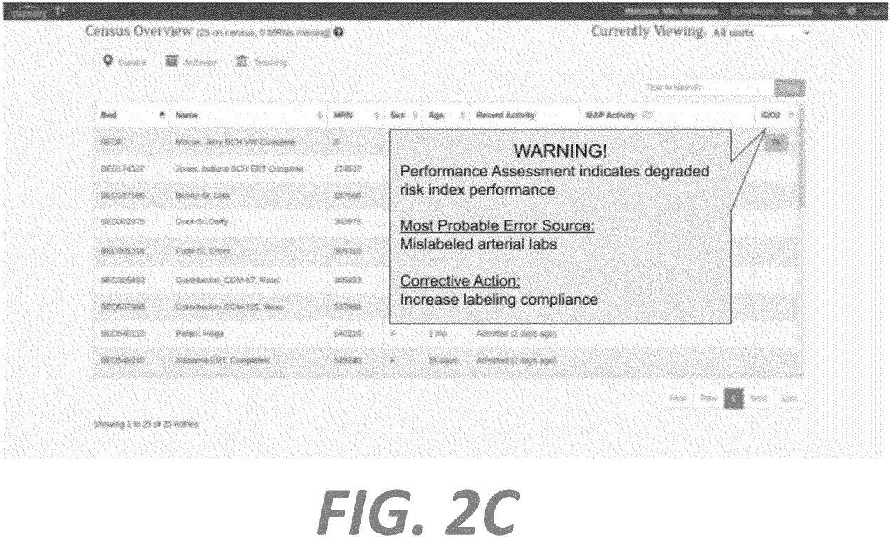

The sensors 102 may include, but are not limited to, a blood oximeter, a blood pressure measurement device, a pulse measurement device, a glucose measuring device, one or more analyte measuring devices, an electrocardiogram recording device, amongst others. In addition, the patient 101 may be administered exams and tests (e.g., “gold standard” tests) and the data may be stored in an electronic medical record (EMR) 103 (shown in FIG. 2A).

Medical devices 102, such as a pulse oximeter, often provide an erroneous output. To determine how close the output of the device 102 is to the actual property to be read (e.g., oxygen saturation), prior art techniques draw blood from the patient 100 and make a direct reading of the oxygen saturation. Although invasive, this is considered a reliable way to precisely determine oxygen saturation. We refer to this type of method as a “gold standard” way of determining the property (in this case, oxygen saturation). To determine the accuracy of the device, one simply compares the gold standard reading (e.g., via the blood test) to the reading on the pulse oximeter for a given time. The difference between those values is the error of the device 102. If that error is too great, the device 102 may be considered defective and potentially produce meaningless or, worse yet, dangerous data (e.g., leading to faulty medical treatments). Various embodiments may store “gold standard” data in the electronic medical record 103, and may furthermore correlate the gold standard data with collected sensor 102 data.

The electronic medical record 103 may include but is not limited to stored information such as hemoglobin, arterial and venous oxygen content, lactic acid, weight, age, sex, ICD-10 code, capillary refill time, subjective clinician observations, patient self-evaluations, prescribed medications, medications regiments, genetics, test results, allergies, etc.

In addition, the patient 101 may be medically coupled to one or more treatment devices 104 that are configured to administer treatments to the patient 101. In some embodiments, one or more treatment devices 104 may be controlled by a system 100 as disclosed herein, for example in response to output defining a patient state or medical condition from a trajectory interpreter module. In various embodiments, the treatment devices 104 may include extracorporeal membrane oxygenator, mechanical ventilator, medication infusion pumps, implantable ventricular assist devices, etc.

Illustrative embodiments provide real-time automatic determination of the performance of a predicted risk index for a particular patient variable or other patient event (collectively referred as calculated internal patient state variables) based at least partially on data acquired directly from the sensors 102 and peripheral devices. The real-time determination is advantageous over prior art methods that rely on retrospective validation of performance criteria. Prior art methods do not dynamically determine root causes of errors encountered in clinical practice, nor are they able to provide insight to the medical practitioner regarding the suspected cause(s) of the error. Additionally, identifying and/or correcting errors in the predicted internal state variables provides practical improvements to the care provided by the medical practitioner. For example, the medical practitioner may make medical decisions based on a calculated internal state variable that meets some threshold performance variable (i.e., when the calculated variable is likely to be accurate). On the other hand, by determining the performance of the calculated internal state variable, medical practitioners may avoid needlessly checking on the patient 101 based on erroneous calculations that indicate a poor patient health state. This can help assist with reducing alarm fatigue, which is problematic in the medical industry. Accordingly, illustrative embodiments lead to enhanced clinical outcomes for the patient 101.

The system 100 may track all of the relevant inputs (e.g., sensor 102 data) used to calculate a given internal state variable over a period of time. Multiple examples of various internal state variables that may be calculated, and relevant inputs are disclosed in U.S. Application No. 17/033,591, which is incorporated herein by reference in its entirety. Illustrative embodiments calculate a probability that the patient 101 is experiencing a particular event or that a patient variable (e.g., biomarker) is above or below a certain critical value for a biomarker as a function of the internal state variable.

FIG. 2A schematically shows details of the system 100 in accordance with illustrative embodiments of the invention. The system 100 includes a sensor/medical device interface 106 configured to communicate with the one or more sensors 102 and/or one or more medical devices 104. For convenience, illustrative embodiments may refer to receiving data from one or more sensors 102, medical devices 102, and/or treatment devices 104 generally as receiving data from the sensors 102. However, it should be understood that discussion of receiving data from the sensors 102 is intended to also include receiving data from the medical devices 104 and does is not limited to receiving data from a dedicated sensing device.

To reiterate, the interface 106 receives/streams real-time patient data from the sensors 102. The interface 106 may receive data from a variety of sensors 102, such as blood oximeter, a blood pressure measurement device, a pulse measurement device, a glucose measuring device, one or more analyte measuring devices, and/or an electrocardiogram recording device, amongst others. In some embodiments, the interface 106 simultaneously communicates with a plurality of sensors 102 and/or medical devices. Accordingly, the interface 106 may aggregate and/or compile the various received patient data.

The system 100 may be configured to receive patient related information, including real-time information from the sensors 102, EMR patient information from the electronic medical record 103, information from the treatment devices 104, such as ventilatory settings, infusion rates, types of medications, and other patient related information, which may include the patient’s medical history, previous treatment plans, results from previous and present lab work, allergy information, predispositions to various conditions, and any other information that may be deemed relevant to make.

To that end, the system 100 includes a database 105 where the received patient data (e.g., including the real-time data received through interface 106) is stored. The database 105 also has access to the patient EMR 103, as described previously. The patient EMR 103 may include information about the race, age, weight, diagnosis, procedures, and other relevant patient information that may be used by the system 100. The database 105 may also communicate with the sensor interface 106 to store the real-time data as it is received via the interface 106.

In addition to streamed data, the database 105 may include information relating to collected gold standard data (e.g., a blood draw) and/or lab data. Frequently, the gold standard data is a lab measurement, but not all lab measurements are gold standard. In various embodiments, gold standard measurements refer to lab measurements that are used as benchmarks against streamed sensor data.

In various embodiments, the database 105 may receive and/or store information relating to hospital unit type (e.g., cardiac ICU, general ICU), the type of hospital, geographic location, and/or other relevant data.

The system 100 may also include a retrospective database 108 that includes the data upon which the predictive indexes were initially based. As an example, the retrospective database 108 may contain measurement data collected on representative patients from other points in time or locations that can be used as reference when examining suspected error conditions in the current data collected by the system 100. The retrospective database 108 may also include previous predictive analytics calculated on similar patient cohorts, which can also be used as referential information when assessing the system 100 performance.

The system of FIG. 2A may include a risk-based monitoring engine 1000 (also referred to as “risk engine 1000”) configured to receive data from bedside monitors 102, electronic medical records 103, treatment devices 104, and any other information that may be deemed relevant to make an informed assessment regarding the patient’s clinical risks, and any combination thereof of the preceding elements.

In illustrative embodiments, the risk engine 1000 may include a physiology observer module 119 that utilizes multiple measurements to estimate probability density functions (PDF) of internal state variables (ISVs) that describe the components of the physiology relevant to the patient treatment and condition in accordance with a predefined (e.g., physiology-based) model of the ISV. The output of the model for the ISV is a probability density of the ISV value. From the probability density of the ISV, a probability that the particular internal state variable exceeds a corresponding pre-defined threshold may be determined (e.g., what is the probability (i.e., risk index) that SvO2 is below 40%). Additionally, other statistical manipulation of the probability density may be used and provided to the medical practitioner (e.g., a mean, median, and/or mode of the ISV value). The ISVs may be directly observable with noise (as a non-limiting example, heart rate is a directly observable ISV), hidden (as a non-limiting example, oxygen delivery (DO2) defined as the flow of blood saturated oxygen through the aorta cannot be directly measured and is thus hidden), or measured intermittently (as a non-limiting example, hemoglobin concentration as measured from Complete Blood Count tests is an intermittently observable ISV).

In some embodiments, when the physiology observer module 119 evaluates a set of ISVs at a given time step (e.g., tₖ; tₖ₊₁; generally tₖ₊ₙ), the system 100 may not have a complete set of ISV measurements contemporaneous with that given time step. For example, the system 100 may have measurements for that given time step for some internal state variables, but may not have measurements for that given time step for some other internal state variables (e.g., a contemporaneous measurement for an intermittent ISV may not be available for the given time step). Consequently, that intermittent ISV is, for purposes of evaluating ISVs at the given time step, a hidden ISV. However, evaluation of the set of ISVs by the physiology observer module 119 (as described herein) is nevertheless possible according to embodiments described herein because the predicted PDFs of ISVs carry in them the influence of past measurements of that intermittent ISV, and consequently those predicted PDFs of ISVs are, in illustrative embodiments, sufficient input for the physiology observer module 119.

In one embodiment, instead of assuming that all variables can be estimated deterministically without error, the physiology observer module 119 of the present disclosure provides probability density functions as an output. Additional details related to the physiology observer module 119 are provided herein.

The clinical trajectory interpreter module 123 may be configured, for example, with multiple possible patient states, and may determine which of those patient states are probable and with what probability, given the estimated probability density functions of the internal state variables. Patient state provides a qualitative description of the physiology of the patient at a particular point of time of the patient’s clinical course, which qualitative description is derived from quantified evidence (e.g., measurements of one or more of the patient’s internal state variables), and which qualitative description is recognizable by medical practice, and may have implications to clinical decision-making. A patient state may be a medical condition, such as an adverse medical condition. The term “patient state” does not include the patient’s state of consciousness (e.g., awake and/or asleep; etc.)

Examples of particular patient states include, but are not limited to, adverse medical conditions such as inadequate delivery of oxygen, inadequate ventilation of carbon dioxide, hyperlactatemia, acidosis; amongst others. Other examples of particular patient states include, but are not limited to, hypotension with sinus tachycardia, hypoxia with myocardial depression, compensated circulatory shock, cardiac arrest, hemorrhage, amongst others. In addition, these patient states may be specific to a particular medical condition, and the bounds of each of the patient states may be defined by threshold values of various physiological variables and data. In various embodiments, the clinical trajectory interpreter module 123 may determine the patient conditions under which a patient may be categorized using any of the information gathered from reference materials, information provided by health care providers, other sources of information. In addition, these patient states may be specific to a particular medical condition, and the bounds of each of the patient states may be defined by threshold values of various physiological variables and data.

The reference materials may be stored in the database 105 or other storage device that is accessible to the risk-based monitoring application via a network interface, for example. These reference materials may include material synthesized from reference books, medical literature, surveys of experts, physician provided information, and any other material that may be used as a reference for providing medical care to patients. In some embodiments, the clinical trajectory interpreter module 123 may first identify a patient population that is similar to the subject patient being monitored. By doing so, the clinical trajectory interpreter module 123 may be able to use relevant historical data based on the identified patient population to help determine the possible patient states.

The clinical trajectory interpreter module 123 is capable of also determining the probable patient states under which the patient can be currently categorized, given the estimated probability density functions of the internal state variables, as provided by physiology observer module 119. In this way, each of the possible patient states is assigned a probability value from 0 to 1. The combination of patient states and their probabilities is defined as the clinical risk to the patient.

Additional details regarding the risk-based patient monitoring engine 1000 and calculation of hidden internal state variables are described in U.S. Pat. Application Nos. 17/033,591, and 17/501,978, each of which is incorporated herein by reference in its entirety.

The system 100 includes a performance assessor 110 configured to assess the performance of the predictive analytics generated by the risk engine 1000. To that end, the performance assessor 110 may calculate a true positive rate and a false positive rate of the predictive analytics. Depending on the internal state variable, the assessor 110 may look at a variety of data sources. For example, when the internal state variable is a predicted event, the assessor 110 looks at the predicted outcome of the event (e.g., event is likely to occur OR the event is not likely to occur) and then looks at the corresponding “gold standard” data (i.e., the event did occur OR the event did not occur). In a similar manner, when the internal state variable is a particular patient variable (e.g., biomarker value), the assessor 110 looks at the predicted outcome of the patient variable (e.g., the biomarker is likely to be above or below a particular threshold value) and the corresponding “gold standard” data (e.g., the biomarker is above or below the particular threshold value). Furthermore, in some embodiments, the predicted outcome for the internal state variable may be a range of the patient variable or a particular value.

The performance assessor 110 also communicates with the retrospective database 108 to obtain expected performance criteria for the risk engine 1000. The performance assessor 110 compares the performance of the system 100, particularly the risk engine 1000, to previous performance on retrospective cohorts in the retrospective database 108. When performance is below a prescribed threshold, the performance assessor 110 communicates with a diagnostic trigger module 116 that is configured to adjust the level of data logging for a deeper diagnostic review. Additionally, the performance assessor 110 may communicate with a notification module 118 configured to alert medical practitioners and/or a system 100 manager that the performance of the system 100 is below the prescribed threshold. Accordingly, medical practitioners may adjust their medical practice (e.g., treatment of the patient) based on this information. In various embodiments, the subscription rules 128 assign different subscription levels to different medical practitioners. For example, the direct care team may be subscription level 1, whereas the management team by may be subscription level 2. The subscription rules may be used in the notification rules. Accordingly, different subscriptions may receive different notifications and/or notifications for different reasons.

In various embodiments, the performance assessor 110 may also include an error source identifier 111 configured to identify the most likely cause(s) for the reduction in the predictive performance of the system 100.

The system 100 may include a quality reporting module 114 that gathers and reports information about the patient specifics for which the derived index has been used for. Some examples of this information include, but are not limited to, number of patients used for, average derived index values, demographic/diagnosis information etc.

Database queries and performance assessment report generation can be performed on regular predefined intervals, such as weekly, monthly, or quarterly. These intervals can be driven based on data collection rates and system usage rates, e.g. only execute the assessment when there is a sufficient number of data to assess performance. The process can also be executed on an on-demand basis, when support staff suspect performance of the predictor index may be out of specification, or when the notification module 118 provides a notification. The specific assessments performed as part of the process can be configured by users either before or after deployment.

The system may include a notification module 118 configured to receive subscription rules and notification rules, as well as patient status information. As the subscription rules are met, the notification module 118 sends a notification to the various subscribed users in accordance with their subscription level. For example, notifications may be sent when system 100 performance falls beneath a given threshold, or goes above a given threshold. Furthermore, the notification module 118 may send information from the reporting module 114 regarding the suspected causes of error.

FIGS. 2B-2C show screenshots of notifications that may be sent to a medical practitioner. These screenshots may be displayed on, among other things, a dedicated display and/or on a mobile device (e.g., smartphone) of the medical practitioner via a web-browser or smartphone application. FIG. 2B shows a screenshot of a notification and warning within a patient view. In the patient view, metrics related to the patient are displayed, as discussed in previous applications incorporated herein by reference. FIG. 2C shows a screenshot of a notification and warning within a census view. The notifications help bring the medical practitioner’s attention to the error condition, and may also display one or more probably error sources, and/or one or more corrective actions. Accordingly, as is discussed further below, various embodiments use the system and methods described herein to determine that an error exists, and the likely cause/source of the error(s). Furthermore, various embodiments instruct the medical practitioner to take a corrective action to correct the error condition. The early and automatic detection and notification of an error condition assists with accurate diagnosis and/or treatment of a patient being monitored (e.g., for performance with a particular protocol, for a particular value on an internal state variable, etc.). Furthermore, by notifying the medical practitioner about the error, the likely source of the error, and the associated corrective action, illustrative embodiments enable real-time improved medical treatments and patient outcomes that may otherwise be unachievable due to delay in corrective action.

Each of the above-described components is operatively connected by any conventional interconnect mechanism. FIG. 2A simply shows a bus communicating each of the components. Those skilled in the art should understand that this generalized representation can be modified to include other conventional direct or indirect connections. Accordingly, discussion of a bus is not intended to limit various embodiments.

Indeed, it should be noted that FIG. 2A only schematically shows each of these components. Those skilled in the art should understand that each of these components can be implemented in a variety of conventional manners, such as by using hardware, software, or a combination of hardware and software, across one or more other functional components. For example, the performance assessor 110 may be implemented using a plurality of microprocessors executing firmware. As another example, the performance assessor 110 may be implemented using one or more application specific integrated circuits (i.e., “ASICs”) and related software, or a combination of ASICs, discrete electronic components (e.g., transistors), and microprocessors. Accordingly, the representation of the components in a single box of FIG. 2A is for simplicity purposes only.

In some embodiments, components of the system may be separated into different components. Additionally, various components, such as the sensor/medical device interface 106 of FIG. 2A, may be distributed across a plurality of different machines - not necessarily within the same housing or chassis.

Additionally, in some embodiments, components shown as separate (such as the risk engine 1000 and the performance assessor 110 in FIG. 2A) may be replaced by a single component. Illustrative embodiments may include additional modules not explicitly shown here. Furthermore, certain components and sub-components in FIG. 2A are optional. For example, some embodiments may not include the non-conformance module 116.

It should be reiterated that the representation of FIG. 2A is a significantly simplified representation of the system 100. Those skilled in the art should understand that such a device may have other physical and functional components, such as central processing units, other packet processing modules, and short-term memory. Indeed, the system 100, in various embodiments, includes one or more of the following: a processor, a memory coupled to the processor, and a network interface configured to enable the system to communicate with other devices over a network. In addition, the system may include a risk-based monitoring application that may include computer-executable instructions, which when executed by a processor, cause the system to be able to afford risk-based monitoring of the patients, such as the patient 101. Accordingly, this discussion is not intended to suggest that FIG. 2A represents all of the elements of the system 100.

As described above, in various embodiments, the risk based monitoring application produces risk indexes whose value correspond to the current level of risk fo r a particular condition or patient event. These risk indexes are developed and tested at least in part based on data previously collected on patients. Thus, development of a risk index is generally performed retrospectively by, e.g., collecting and processing data from thousands of patients. The indexes are back tested against the retrospective cohorts to validate that they work on these cohorts with data that has already been collected. In many instances, the patient cohorts are broad and intended to be representative (e.g., may include data from 10 different hospitals or more). However, even with this large pool of patient data, the indexes performance is generally tested only against certain scenarios, certain clinical settings, certain specific hospital settings, and those given patient populations and the treatment protocols in those hospitals.

Various embodiments practically apply the risk indexes by validating and ensuring their performance in real clinical settings, where the demographics and types of patients may vary, and/or the protocols used by the hospitals may vary. Accordingly, illustrative embodiments described herein enable performance based validation and correction of risk indexes, e.g., particularly to new medical institutions (i.e., not used in the initial retrospective cohort for development of the indexes). In various embodiments, the system is configured so that the risk indexes performance are within a desirable range at a new center as on the other retrospective cohorts.

Illustrative embodiments provide a continuous assessment of the predictive risk indexes after deployment. The continuous assessment allows the hospital or medical facility to determine if the performance of the risk indexes (i.e., the ability of the system to accurately predict the patient variable or event) meets a certain expected performance criteria (i.e., are the indexes within specification). It should be understood by one skilled in the art that the continuous monitoring of predictive risk indexes in a dynamic environment (e.g., new hospital where the system has not previously received data) provides a practical application that results in corrective action of what may otherwise be a misdiagnosed or undiagnosed condition, and/or an erroneous hidden internal state variable calculation. Various embodiments furthermore may determine, if there is a significant change in performance of the risk indexes, the reason for the change (e.g., new procedure, new sensors, new surgeon, patients coming from a different part of the world, demographic differences, etc.).

In various embodiments, the system 100 can be installed locally within a hospital network, or in a remote computational cloud. It can operate on a single computational server or it can operate over multiple servers and communicate between servers via a network protocol such as HTTP.

It should be apparent that providing updated performance characteristics “in the field” provide a number of advantages, including use of the metric(s) as a diagnostic (e.g., to determine the source of an error) and as an actionable item (e.g., adjust the location of a sensor, replace a faulty sensor, etc.). Furthermore, medical practitioners may choose to rely on data provided by the system, or not, based on the performance characteristics of the system as tested in real time.

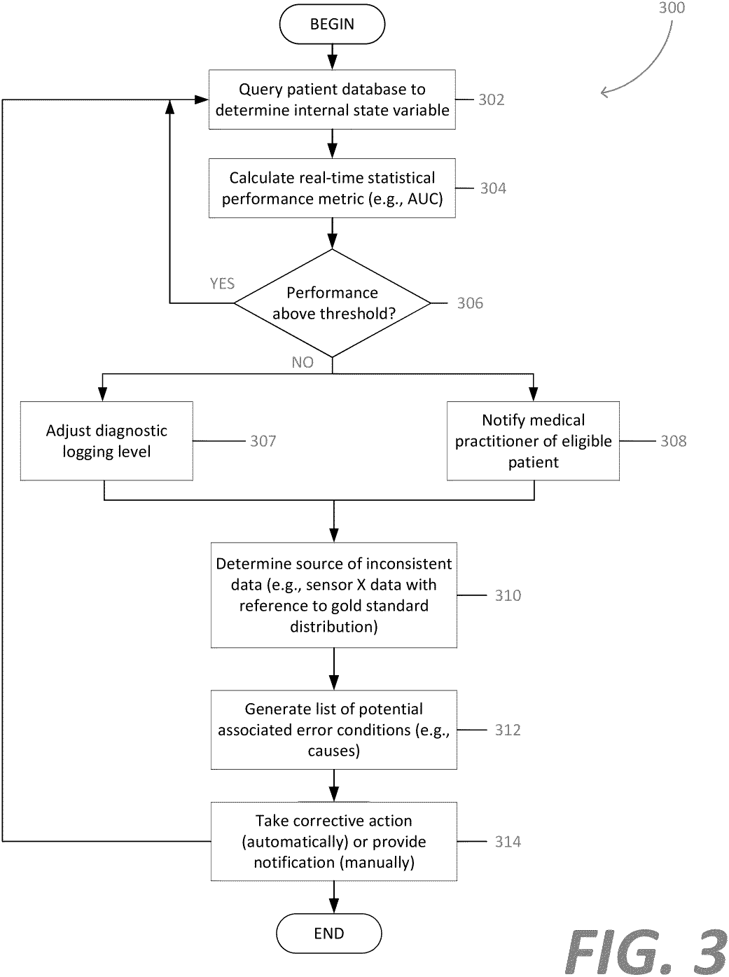

FIG. 3 shows a process 300 of determining that performance of a predictive analytics system 100 in accordance with illustrative embodiments of the invention. It should be noted that this process can be a simplified version of a more complex process that may normally be used. As such, the process may have additional steps that are not discussed. In addition, some steps may be optional, performed in a different order, or in parallel with each other. Accordingly, discussion of this process is illustrative and not intended to limit various embodiments of the invention. Finally, although this process is discussed with regard to assessing the performance of a single analytics system 100, the process of FIG. 3 can be expanded to cover processes for assessing the performance of a plurality of analytics systems 100 at the same time. Accordingly, the process 300 is merely exemplary of one process in accordance with illustrative embodiments of the invention. Those skilled in the art therefore can modify the process as appropriate.

The process begins at step 302, which queries the patient database 105 to determine an internal state variable. In some embodiments, a plurality of internal state variables may be determined. The internal state variable (or “ISV”) is a parameter of the patient’s physiology that is physiologically relevant to treatment and/or a condition of a patient. In various embodiments, the internal state variable may be represented by a model developed based on human physiology (e.g., using known physiological relationships) and/or data (e.g., from a machine learning model receiving a large sample data set). The internal state variable may be calculated using the received patient data and the model. In various embodiments, the output of calculating the internal state variable is a probability or a probability density relating to the internal state variable (e.g., the value or the state of the ISV). Thus, among other things, the output of calculating the internal state variable may include a particular probability density for a patient variable (e.g., PaCO2 value probability density), or a probability that the patient is experiencing a particular health event. Among other ways, the output may be a probability or probability density representing: an estimated value for a particular variable (e.g., PaCO2 value), that the value for the variable is above or below a threshold (e.g., PaCO2 value is greater than 50 mmHg), that the value for the variable is within a particular range (e.g., PaCO2 is between 45 mmHg and 55 mmHg), and/or that a particular health event is occurring or not occurring (e.g., is the patient experiencing respiratory failure).

ISVs may be directly observable with noise (as a non-limiting example, heart rate is a directly observable ISV), hidden (as a non-limiting example, oxygen delivery (DO2) defined as the flow of blood saturated oxygen through the aorta cannot be directly measured and is thus hidden), or measured intermittently (as a non-limiting example, hemoglobin concentration as measured from Complete Blood Count tests is an intermittently observable ISV). Other examples of ISVs include, without limitation, Pulmonary Vascular Resistance (PVR); Cardiac Output (CO); hemoglobin, and rate of hemoglobin production/loss.

Additionally, in some embodiments, the relevant patient data may be indirectly received (e.g., may not be directly observable/measurable). A hidden Internal State Variable, means an ISV that is not directly measured by the sensor 102 coupled to the patient 101. Some hidden ISVs cannot be directly measured by the sensor. In some embodiments, the module may receive, in addition to or instead of sensor data, data representing a risk that the patient is suffering a specific adverse medical condition as indicated by the probability of the hidden internal state variable being in a particular state. Some hidden ISVs require, for example, laboratory analysis of a sample (e.g., “gold standard” blood sample) taken from the patient. Additional details regarding hidden internal state variables are described in U.S. Pat. Application No. 17/033,591, which is incorporated herein by reference in its entirety.

The patient database 105 includes data from the various sensors 102, devices 104, EMR 103, and patient labs, among other things. The internal state variable is calculated based on a model developed using retrospective cohort data. The internal state variable may be calculated for one or more patients over a given period of time. In various embodiments, the internal state variable that is calculated may be a hidden internal state variable.

To determine the internal state variable, the process may receive patient data directly or indirectly. The patient data may be received directly (e.g., streamed) in real-time from the sensors 102 and/or medical devices.

Among other things, the received patient data may include: expired CO2, end-tidal CO2 measurement (EtCO2), minute ventilation, ventilator mode, drug infusion rate, respiratory rate, PaCO2 arterial blood gases, Hb Hemoglobin, HR Heart Rate, SpvO2 Pulmonary Venous Oxygen Saturation, SaO2 Arterial Oxygen Saturation, SvO2 Systemic Venous Oxygen Saturation, SpO2 Pulmonary Venous Oxygen Saturation, Mean Arterial Blood Pressure, Central Venous Pressure, Left Atrial Pressure, Right Atrial Pressure, patient weight, patient age, patient height, and/or patient medical history.

The process then proceeds to step 304, where a statistical performance metric is calculated by the assessor 110 based on the predicted internal state variable and gold standard data obtained for the patients over the same given period of time. The performance measurement may be performed at any given time (e.g., in real time on the most recent data collected by the system). This performance metric may also be stored in the patient database 105. Although various embodiments describe that the performance metric is for a predicted internal state variable, in some embodiments, the performance metric may be for the operation of a sensor 102. Because multiple data points are streamed from the sensor, and corresponding reliable gold standard data (e.g., blood draw data) is obtained, the system 100 may calculate performance metrics regarding the sensor.

As described previously, when the internal state variable is a predicted event, the assessor 110 looks at the predicted outcome of the event (i.e., event is likely to occur OR the event is not likely to occur) and then looks at the corresponding “gold standard” data (i.e., the event did occur OR the event did not occur). In a similar manner, when the internal state variable is a particular patient variable value (e.g., biomarker value), the assessor 110 looks at the predicted outcome of the patient variable (i.e., the patient variable is likely to be above or below a particular threshold value) and the corresponding “gold standard” data (i.e., the patient variable is above or below the particular threshold value). Furthermore, in some embodiments, the predicted outcome may be a range of the patient variable or a particular value.

Thus, for example, at time tₖ, the database 105 may include data relating to a sensor measurement and a corresponding gold standard lab measurement. The database 105 may also have similar paired data at a plurality of other times, tₖ₊₁... tₖ₊ₙ. Furthermore, similar paired data sets may be obtained for a plurality of patients.

Using this information, a receiver operating characteristic (ROC) curve may be generated, and an area under the curve (AUC) may be determined. Those skilled in the art understand how to create and interpret an ROC graph. The ROC is a graphical plot that illustrates the diagnostic ability of the risk engine 1000 as its discrimination threshold is varied.

For the sake of discussion, the examples discussed below refer to the statistical performance metric of the ROC curve and the AUC. However, it should be understood that various embodiments may use a variety of different statistical performance metrics (e.g., root mean square), and are not limited to the disclosed performance metrics.

The process then proceeds to step 306, which asks if the performance of the system calculated in step 304 is above a given threshold. As an example, the performance may be judged based on a particular AUC.

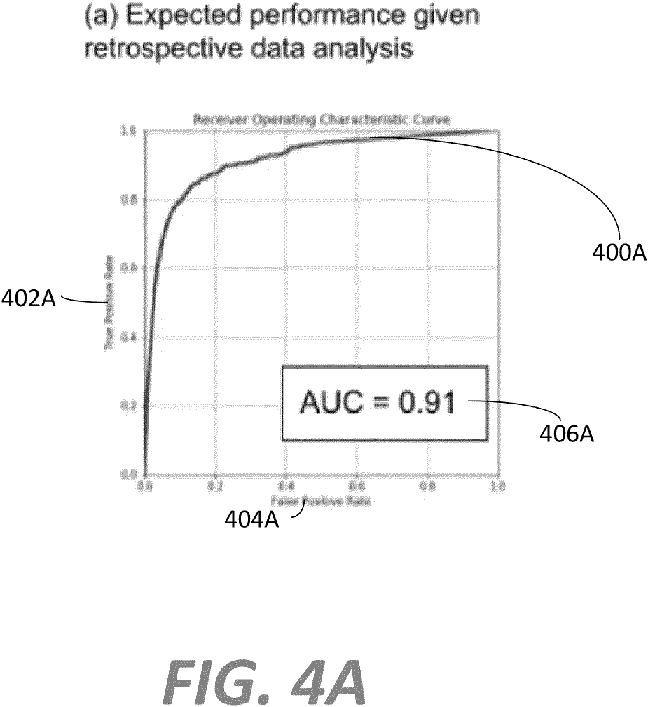

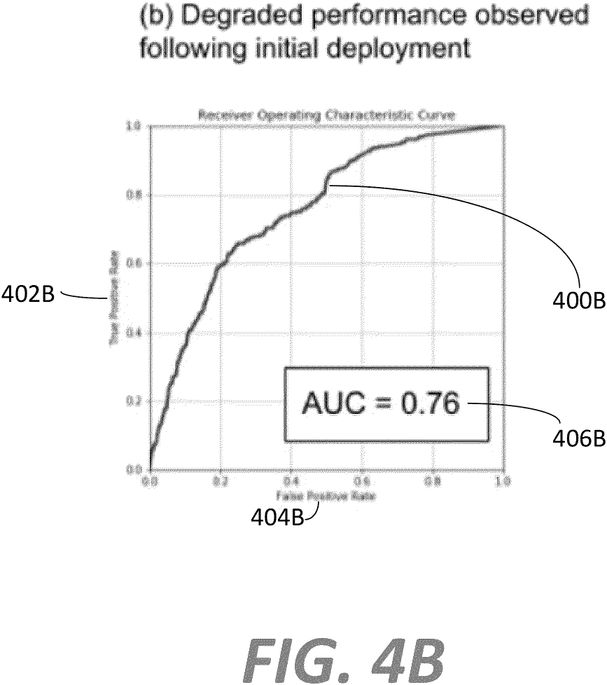

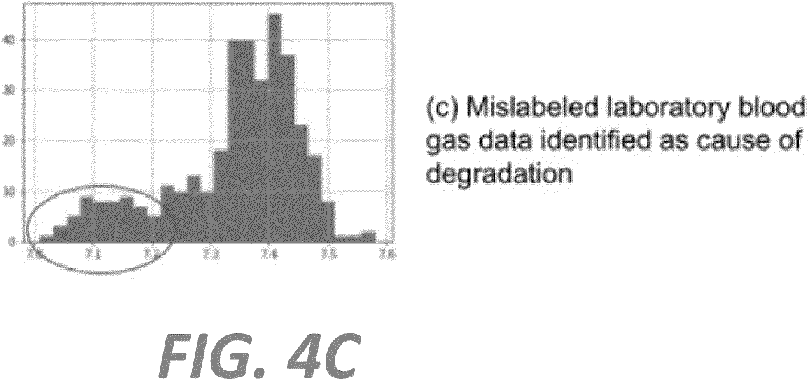

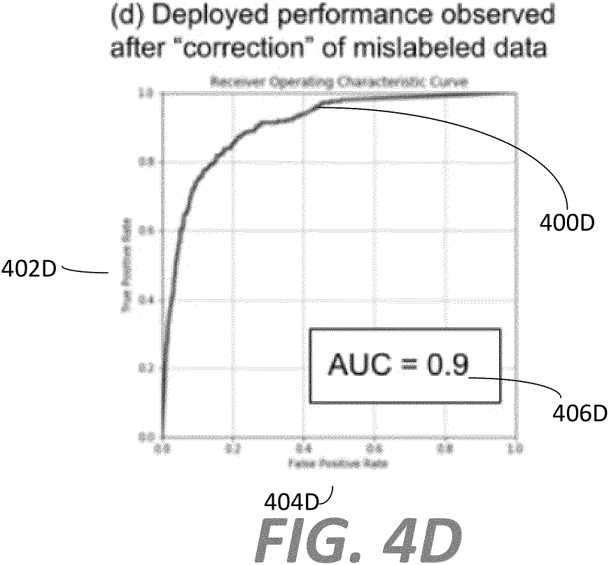

FIG. 4A schematically shows the ROC curve 400A that may be used as a comparative performance assessment threshold in step 306, in accordance with illustrative embodiments. The ROC curve 400A may be developed using a retrospective cohort (e.g., from data stored in the retrospective database 108) for a given predicted internal state variable. As the predictive indexes are developed using a retrospective cohort, the model may be refined until a particular performance criteria is reached. In this example, the acceptable performance criteria is the AUC 406A having a value of 0.91.

As known by those skilled in the art, the ROC curve 400A is created by plotting the true positive rate 402 against the false positive rate 404 at various threshold settings. The true positive rate 402A is also known as sensitivity, recall, or probability of detection. The true positive rate may be calculated as:

$$T r u e P o s i t i v e R a t e = \frac{T r u e P o s i t i v e s}{T r u e P o s i t i v e s + F a l s e P o s i t i v e s}$$

The false positive 404 rate is also known as probability of false alarm and can be calculated as (1 - specificity). The false positive rate may be calculated as:

$$F a l s e P o s i t i v e R a t e = \frac{F a l s e P o s i t i v e s}{F a l s e P o s i t i v e s + T r u e N e g a t i v e s}$$

In various embodiments, the retrospective cohort based ROC curve 400A may be provided to the system 100 and/or may be generated using patient data from another healthcare facility or site from the ROC curve 400B generated in step 304.

FIG. 4B schematically shows the ROC curve 400A generated by the performance assessor 110 using data from the patient database 105. FIG. 4B thus displays the performance of the risk analytics engine 1000 (referred to as the risk engine 1000) “in the field” on real patients. If the performance of the risk engine 1000 (also referred to as the performance index) is greater than the given threshold, then the process 300 returns to step 302. However, in this example, the performance metric AUC 406B is 0.76, which is beneath the threshold of 0.91.

Returning to FIG. 3, when the performance criteria is beneath the threshold, the process optionally proceeds to step 307, which adjusts the diagnostic logging level. In other words, the amount of data collected for detailed diagnostics is increased. This data may include, but is not limited to, additional information regarding the particular type of sensors that are providing data and the data collection methodology, additional internal information about the current predictive analytics calculations related to the ISV model, and/or additional patient specific information, such as diagnosis, procedures, medications, and demographics, etc.

Simultaneously with step 307, the process may proceed to step 308, which notifies the medical practitioner of the drop in performance. The medical practitioner may be part of the subscription rules and notification rules. For example, the medical practitioner may be the nurse responsible for the patient 101, and also may be working a shift when the patient 101 becomes eligible. To that end, the system 100 may provide through the user interface 112 a notification that shows performance criteria (as it is determined by step 304).

Although not explicitly shown in the process 300 of FIG. 3, it should be understood that throughout the process notifications may be provided to relevant medical staff. To that end, the notification module 118 may receive the subscription rules and the notification rules, as well as system 100 performance metrics. As the subscription rules are met, the notification module 118 sends a notification to the various subscribed users in accordance with their subscription level. For example, notifications may be sent when eligibility status is changed, when the course of action begins (also referred to as protocol enrollment), and/or when the patient is out of compliance.

Furthermore, different subscription levels may receive different notification types. For example, “notification 1” may include a passive notification, such as an alert through the user interface 112; “notification 2” may include an escalated passive notification, such as a text and/or an email; and “notification 3” may include an active notification such as paging the medical staff.

The process then proceeds to step 310, which determines the source of the inconsistent data causing the performance decrease. This may be accomplished by comparing the patient data with the gold standard data for each of the various data sources used to predict the internal state variable. In contrast to steps 304-306, which look at the data at a statistical performance level, step 310 looks at specific instances of erroneous patient data.

For example, the risk engine 1000 may calculate an internal state variable using patient data gathered by a pulse oximeter and blood gas measurements. Pulse oximeters use light absorption to provide a value for oxygen content in arterial blood. Pulse oximeter data is validated by medical device manufacturers using volunteers and partially asphyxiating them (reduced oxygen in breathable air) as they can regulate how much oxygen the volunteers have in their blood. Arterial “gold standard” blood samples are drawn and compared to the pulse oximeter values to validate the results of the pulse oximeter.

When the system 100 is deployed in the field, it receives pulse oximeter data continuously. Every time a practitioner draws blood to obtain the arterial saturation value, which is the gold standard, the blood draw has the corresponding pulse oximeter data for at least one moment in time (i.e., the pulse oximeter data is streamed at the same time as the blood draw). If the patient data from the sensor is inconsistent with the gold standard data, illustrative embodiments may determine that the sensor data is the source (or at least one of the sources) for the performance decrement.

Taking the same example from above, alternatively, it may be that the sensor data is within specification of the gold standard data. Instead, it may be determined that mislabeled laboratory blood gas measurements are the source of the inaccuracy (i.e., venous blood gas samples were labeled as arterial blood gas samples, and vice-versa). FIG. 4C schematically shows an example of a histogram of this mis-labeled blood gas data. The mislabeled blood gas data points are circled. Similar to other examples described above, this error may be determined by the assessor 110 by comparing the patient venous blood samples with a distribution of retrospective cohort patient venous blood samples (e.g., from the retrospective database 108).

The process then proceeds to step 312, which generates a list of potential associated error conditions. Thus, for any given source of error, the system 100 (e.g., an error source identifier 111) looks at associated demographic data (e.g., race, age, weight), associated medical data (e.g., diagnosis, procedure, other patient data such as a heart rate at the time of erroneous measurement) for each of the patients that have a source of error. Additionally, or alternatively, it may be the case that the type of hospital, geographic location of the medical care facility, and/or environmental factors are correlated with a particular error in a sensor. In various embodiments, the list of potential associated error conditions may be provided in a ranked order, i.e., corresponding to most likely to least likely to cause the performance degradation.

The system 100 automatically looks at the performance of various devices, under different operating conditions, for various patients, race, gender, age, patient population, unit type (cardiac ICU, general ICU), type of hospital, geographic location, etc. As an example, pulse oximeter data is unreliable for African American patients when values reach a particular threshold (e.g., are in the 80-90%).

In cases where a plurality of overlapping corresponding conditions exist, the system may generate a probability of the cause of the error (e.g., 90% weight, 70% race, 25% wrong position of sensor, 15% confusion of laboratory gold standard measurements). The system is able to determine the cause of the error because of data comparisons from the retrospective database 105. For example, NIRS pulse oximeters are placed in different locations based on hospital. Some hospitals may place on the cranium, some on the flank, and depending on where the sensor is positioned, the information the sensor provides can vary. If the system assumes a certain placement of the sensor probes, different positions are inconsistent with what the model expects for that data. The distribution of this data is different from what is expected, and the system is able to assign some probability that the cause of the error is the positioning of the sensor 102.

There are several methods for determining the possible causes of error in sensor 102 information, but in general, illustrative embodiments compare the current output data provided by the sensor 102 with a reference signal or set of signals and additional data. As a specific non-limiting example, to identify a list of probable causes of error, the system 100 (e.g., identifier 111) may:
Compiles the distribution of the data provided by a sensor, or information source, e.g., NIRS measurement data, or blood gas lab panel values
The system then compares this distribution with the distribution of a reference set of typical data from this sensor or information source. The comparison can be done using a number of statistical tests, such as a K-S test
If there are multiple sensors or information sources connected, the system can repeat the above process for each additional sensor
Based on the results of the comparisons, the sensors can be assigned a score that indicates how in disagreement with the reference data the data the sensor is providing is.
Based on the score, the sensors can then be ranked in order of most disagreement to least disagreement, with the top of the ranking being the most probable causes of error.
Furthermore, if any particular sensor is in significant disagreement with the reference, the comparison for that particular sensor can be repeated but with finer granularity, comparing to more specific reference data, e.g. only NIRS data collected the cerebrum, or SpO2 data collected from particular demographics of patients. Based on these comparisons, the system can refine the probable cause of the error into a more specific type of error, e.g. faulty NIRS sensor placement.

Other examples of errors that may be identified by various embodiments include, but are not limited to, the use of sidestream capnography to assesses End-tidal Carbon dioxide levels (e.g., as opposed to mainstream capnographythe former is more error prone), the mislabeling of the source of blood gas panels (e.g., venous instead of arterial and vice-versa), the faulty pressure measurements provided by invasive catheter lines while the line is being used for other purposes than measuring pressure (e.g., drawing blood or administering medications), and the impact of medications on data collected from patients (e.g. certain medications can cause unexpected hemodynamic responses).

The process then proceeds to step 314, in which the system takes corrective action automatically, or provides a notification to a medical practitioner to manually take corrective action. For example, if the system has a strong confidence that the cause of the error condition for a particular source of inconsistent data is that the gold standard laboratory measurements were mislabeled (e.g., venous blood gas samples were labeled as arterial blood gas samples, and vice-versa), the system may automatically relabel the data. The process may then optionally return to step 302, and rerun the process using the corrected data to determine the accuracy of the risk engine 1000.

Additionally, or alternatively, the notification module 118 may send a notification to the medical practitioner to take corrective action. For example, the notification may instruct the medical practitioner that the labs are mislabeled, and that they should change the labels. As another example, the system may notify the medical practitioner that it is likely that a particular sensor 102 has not been applied to the patient 101 in an appropriate manner or location.

The process then comes to an end. Although the process comes to an end, it should be understood that the process may be repeated using the results of the corrective action. For example, after corrective action is taken, the process optionally returns to step 302. The patient database 105 is queried using the corrected data, and then a real-time statistical performance metric is calculated at step 304.

FIG. 4D schematically shows an updated ROC curve 400D. When the process returns to step 304, the AUC is calculated as 0.9, which is significantly increased from the previous AUC of 0.76 shown in FIG. 4B.

Illustrative embodiments calculate performance metrics (e.g., accuracy of the medical devices), draw conclusions about how patient data impacts the performance metrics, and may take corrective action. For example, the medical device data may be adjusted and/or recalibrated to make it more accurate.

It should be apparent from the above disclosure that although illustrative embodiments are described as working with a predictive “risk index,” various embodiments are not limited to performance assessment of “risk indexes.” Indeed, various embodiments may operate with any predictive score that is calculated based on medical data and predicts an underlying event, condition or patient variable in a medical setting for which a gold standard or reference “true value” can be collected.

The system 100 can be coupled to one or more medical devices 104 and/or sensors 102 (e.g., electrodes) configured to provide therapy to the patient (e.g., therapy electrodes as described above). For example, the system 100 can include, or be operably connected to, circuitry components that are configured to generate and provide a therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuit and under control of one or more processors (e.g., processor) to provide, for example, one or more pacing or defibrillation therapeutic pulses.

The patient database 105 and/or retrospective database 108 can include one or more of non-transitory computer readable media, such as flash memory, solid state memory, magnetic memory, optical memory, cache memory, combinations thereof, and others. The data storage 105 can be configured to store executable instructions and data used for operation of the system 100. In certain implementations, the data storage can include executable instructions that, when executed, are configured to cause the processor to perform one or more functions.

In some examples, the user interface 112 can facilitate the communication of information between the system 100 and one or more other devices or entities over a communications network. For example, where the system 100 is included in a mobile device, the user interface 112 can be configured to communicate with a remote computing device such as a remote server or other similar computing device. The user interface 112 can include communications circuitry for transmitting data in accordance with a Bluetooth® wireless standard for exchanging such data over short distances to an intermediary device(s) (e.g., a base station, a “hotspot” device, a smartphone, a tablet, a portable computing device, and/or other devices in proximity of the patient). The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi™ communications link based on the IEEE 802.11 standard.

In certain implementations, the user interface 112 can include one or more physical interface devices such as input devices, output devices, and combination input/output devices and a software stack configured to drive operation of the devices. These user interface elements may render visual, audio, and/or tactile content. Thus the user interface 112 may receive input or provide output, thereby enabling a user to interact with the system 100.

The system 100 can also include at least one battery configured to provide power to one or more components integrated in the system 100. The battery can include a rechargeable multi-cell battery pack. In one example implementation, the battery can include three or more 2200 mAh lithium ion cells that provide electrical power to the other device components within the system 100. For example, the battery can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the system 100.

The sensor interface 106 can be coupled to one or more sensors configured to monitor one or more physiological parameters of the patient. The sensors 102 may be coupled to the system 100 via a wired or wireless connection. The sensors 102 can include one or more electrocardiogram (ECG), heart vibrations sensors, and tissue fluid monitors (e.g., based on ultra-wide band radiofrequency devices).

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, programmable analog circuitry, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

In some implementations, the processor includes one or more processors (or one or more processor cores) that each are configured to perform a series of instructions that result in manipulated data and/or control the operation of the other components of the system 100. In some implementations, when executing a specific process (e.g., cardiac monitoring), the processor can be configured to make specific logic-based determinations based on input data received, and be further configured to provide one or more outputs that can be used to control or otherwise inform subsequent processing to be carried out by the processor and/or other processors or circuitry with which processor is communicatively coupled. Thus, the processor reacts to specific input stimulus in a specific way and generates a corresponding output based on that input stimulus. In some example cases, the processor can proceed through a sequence of logical transitions in which various internal register states and/or other bit cell states internal or external to the processor may be set to logic high or logic low. As referred to herein, the processor can be configured to execute a function where software is stored in a data store coupled to the processor, the software being configured to cause the processor to proceed through a sequence of various logic decisions that result in the function being executed. The various components that are described herein as being executable by the processor can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

As used in this specification and the claims, the singular forms “a,” “an,” and “the” refer to plural referents unless the context clearly dictates otherwise. For example, reference to “the patient” in the singular includes a plurality of patients, and reference to “the database” in the singular includes one or more databases and equivalents known to those skilled in the art. Furthermore, reference to a plurality is also intended to encompass a singular in various embodiments. For example, reference to “internal state variables” includes one or more internal state variables. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways. For example, it is to be understood that the present disclosure contemplates that, to the extent possible, one or more features of any embodiment can be combined with one or more features of any other embodiment.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the above discussion discloses various exemplary embodiments of the invention, it should be apparent that those skilled in the art can make various modifications that will achieve some of the advantages of the invention without departing from the true scope of the invention.

What is claimed is:

1. A method of determining an internal state variable, the method comprising:
receiving patient data and a model of an internal state variable;
determining the internal state variable using the patient data and the model of the internal state variable;
receiving gold standard data corresponding to the internal state variable;
performing a statistical performance assessment of the model of the internal state variable;
determining whether a performance of the model of the internal state variable is above a prescribed performance threshold; and
determining a source of inconsistent data negatively impacting the performance of the model of the internal state variable.

2. The method as defined by claim 1, further comprising:
generating a list of potential associated error conditions causing the inconsistent data from the source.

3. The method as defined by claim 1, further comprising:
taking corrective action to reduce an error condition causing the inconsistent data from the source.

4. The method as defined by claim 3, further comprising:
repeating the steps of claim 1 after taking the corrective action.

5. The method as defined by claim 1, wherein the model of the internal state variable is based on retrospective data.

6. The method as defined by claim 1, wherein the internal state variable is a particular health event.

7. The method as defined by claim 1, wherein the internal state variable is a particular patient variable.

8. The method as defined by claim 1, wherein the internal state variable is a hidden internal state variable.

9. The method as defined by claim 1, wherein the source of error is a patient characteristic.

10. The method as defined by claim 1, wherein the source of error is a patient characteristic when used with a particular sensor.

11. A system for determining an internal state variable, the system comprising:
a risk based patient monitoring engine configured to calculate an internal state variable using patient data and a model of the internal state variable for a patient;
a retrospective database having gold standard data corresponding to the internal state variable;
a performance assessor configured to:
perform a statistical performance assessment of the model of the internal state variable;
determine whether a performance of the model of the internal state variable is above a prescribed threshold; and
determine a source of inconsistent data negatively impacting the performance of the model of the internal state variable.

12. The system as defined by claim 11, further comprising:
an error source identifier configured to generate a list of potential associated error conditions causing the inconsistent data from the source.

13. The system as defined by claim 11, further comprising:
a notification module configured to report that the performance of the model is below the prescribed threshold, the notification module further configured to report corrective actions that reduce an error condition causing the inconsistent data from the source.

14. The system as defined by claim 11, wherein the internal state variable is a particular health event.

15. The system as defined by claim 11, wherein the internal state variable is a particular patient biomarker.

16. The system as defined by claim 11, wherein the internal state variable is a hidden internal state variable.

17. The system as defined by claim 13, further comprising a display that displays the notification.

18. A computer program product for use on a computer system for determining an internal state variable, the computer program product comprising a tangible, non-transient computer usable medium having computer readable program code thereon, the computer readable program code comprising:
program code for receiving patient data and a model of an internal state variable;
program code for calculating the internal state variable using the patient data and the model of the internal state variable;
program code for receiving gold standard data corresponding to the internal state variable;
program code for performing a statistical performance assessment of the model of the internal state variable;
program code for determining whether a performance of the model of the internal state variable is above a prescribed threshold; and
program code for determining a source of inconsistent data negatively impacting the performance of the model of the internal state variable.

19. The computer program product of claim 18, further comprising:
program code for generating a list of potential associated error conditions causing the inconsistent data from the source.

20. The computer program product of claim 18, further comprising:
program code for taking corrective action to reduce an error condition causing the inconsistent data from the source.
